(19) United States
(12) Patent Application Publication
(10) Pub. No.: US 2006/0088556 A1
(43) Pub. Date: Apr. 27, 2006

(54) Topical formulation and use of buspirone

(76) Inventors: David Cavalla, Cambridge (GB); Robert William Gristwood, Cambridge (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/549,032

(22) PCT Filed: Mar. 22, 2004
(86) PCT No.: PCT/GB04/01242

§ 371 Date: Nov. 9, 2005

(30) Foreign Application Priority Data
Mar. 25, 2003 (GB) 0306859.0

(51) Int. Cl. A61K 9/00

(52) U.S. Cl. 424/400

(57) ABSTRACT

A liquid or semi-solid topical formulation of buspirone, when applied to human epidermis in vitro (following methods defined herein), results in a transepidermal flux rate of buspirone (individual or mean data) in one or more of the following ranges: 0.1 to 1.1 μg/cm²/hour as assessed over 5 hours following application; 0.09 to 0.60 μg/cm²/hour as assessed over 13 hours following application; 0.09 to 0.48 μg/cm²/hour as assessed over 24 hours following application; 0.08 to 0.46 μg/cm²/hour as assessed over 30 hours following application; and 0.08 to 0.39 μg/cm²/hour as assessed over 48 hours following application. Buspirone is useful for the manufacture of a topical medicament for use in the treatment of pruritus or an immune-related skin disease.

### FIELD OF THE INVENTION

This invention relates to topical formulations of buspirone for the treatment of immune-related skin diseases and pruritis.

### BACKGROUND OF THE INVENTION

Buspirone, i.e. 8-[4-[4-(2-pyrimidyl)-1-piperazinyl]butyl]-8-azaspiro[4.5]decane-7,9-dione, is currently used clinically as an anxiolytic. For this purpose, the compound is administered orally. A patch preparation of buspirone for transdermal administration has also been in development for central nervous system-related diseases.

Buspirone is also being developed as an agent for the treatment of pathological condition associated with immune responses. This utility, and its topical and systemic use, are described in U.S. Pat. No. 5,484,788, U.S. Pat. No. 5,631,017 and WO 94/22448. These patent specifications disclose the ability of buspirone to inhibit oxazolone hypersensitivity reactions in mice.

U.S. Pat. No. 5,637,314 discloses the topical and systemic application of buspirone or a derivative thereof for treating atopic dermatitis, and presents clinical data from an oral trial with buspirone. In general, systemic administration provides the desired effect after some considerable time.

In particular, U.S. Pat. No. 5,484,788 refers to systemic and topical administration of buspirone, in order to obtain immunosuppression. No effective dosage for topical administration is given, but it is stated that the amount will be lower than if given systemically.

Data from studies in mice of the oxazolone hypersensitivity reaction are reported by McAloon et al., 1995, Int. Arch. Allergy Immunol., 107, 437438. From this publication, it is possible to conclude that buspirone applied topically (in an undefined solution) inhibits oxazolone ear swelling over the range 100 mg/ml to 0.25 mg/ml.

Pruritus (itching) is an unpleasant sensation that elicits the desire to scratch. It is a distressing symptom that can cause discomfort and threaten the effectiveness of the skin as a major protective barrier. Because of the subjective nature of pruritus, the lack of a precise definition, and the lack of suitable animal models, pruritus is a disorder that has not been researched adequately.

The skin comprises 15% of the body's total weight, and is the largest organ of the body. The skin has significant psychosocial and physical functions. Its function as a protective mechanism is the skin's most important role. But skin is also essential to self-image and one's ability to touch and be touched, thereby providing an important component of communication.

Symptoms of generalized itching, without rash or skin lesions, may be related to anything from dry skin to an occult carcinoma, and the etiology of the symptoms should be explored. Common non-malignant etiologic factors include drug reactions, xerosis, scabies, or primary skin diseases. Pruritus is one of the most common complaints of the elderly patient, but estimates of the significance of pruritic symptoms in the elderly population vary from 10% to 50%. The most common diagnosis related to pruritus in this population is simply dry skin.

Generalized pruritus is found in about 13% of all individuals with chronic renal disease and about 70%-90% of those undergoing hemodialysis for its treatment. Cholestatic liver disease with intrahepatic or posthepatic obstruction, with or without increased serum levels of bile acids, is often associated with pruritus. Other etiologic factors include (but are not limited to) primary biliary cirrhosis, cholestasis related to phenothiazines or oral contraceptives, intrahepatic cholestasis in pregnancy, and posthepatic obstruction.

### SUMMARY OF THE INVENTION

The present invention is based at least in part on the finding that buspirone may have valuable properties following topical administration to man, e.g. for the treatment of immune-related skin diseases (for example atopic dermatitis and psoriasis) or pruritis. In particular, the invention can provide acute relief (following a single application) from the symptoms of pruritis, and this is a major advantage over any treatment that addresses only longer-term treatment.

For efficacy in such conditions (immune-related skin diseases and pruritis), skin penetration of buspirone is required. It will be appreciated that penetration through the skin of the mouse ear, as evident from McAloon et al., will not be directly comparable to penetration through human skin. In general, penetration through human skin is substantially dependent on penetration through the stratum corneum, which is the outermost layer. There are multiple variables which will determine the delivery of buspirone from a particular formulation to the affected area, including the concentration of buspirone, the presence of penetration enhancers and/or other agents that modify the kinetics of buspirone skin penetration. The rate at which buspirone penetrates through the skin is important as an effective formulation should deliver sufficient buspirone for efficacy but insufficient to cause symptoms of buspirone overdosage. The area of skin to be treated must also be taken into consideration, particularly when considering symptoms of buspirone overdosage (see Physician's Desk Reference edition 2000 page 822 published by Medical Economics Company Inc, Montvale N.J., USA). These variables can be controlled by one of ordinary skill in the art, once it has been appreciated that the desired endpoint can be achieved.

The optimal epidermal flux rate for topically administered buspirone in the treatment of immune-related skin diseases and pruritis has not been previously defined. Nor has it previously been appreciated that, when administered topically in a suitable dose, buspirone can be effective to treat, not only immune-related conditions but also pruritis, including that associated with non-immune related conditions.

### DESCRIPTION OF THE INVENTION

The active agent for use in the invention is typically a buspirone salt such as the hydrochloride. The term “buspirone” is used herein to refer to any active form of the compound.

This specification defines the optimum flux rate of buspirone through human skin defined in an in vitro test system such that a topical formulation with this characteristic will produce effects in the therapeutic range for the treatment of immuno-related disorders and pruritis. This optimal flux rate may be achieved using a number of different topical formulations.

Preferred formulations are non-occlusive, and liquid or semi-solid. Application, e.g. by rubbing, may be made to the whole area, e.g. 20, 50, 100 or more cm², of the topical symptoms. This area may comprise substantially all of the body, or at least a part, e.g. a limb, thereof.

To prepare a topical formulation, a therapeutically effective concentration of the compound is placed in a dermatological vehicle as is known in the art. The amount of the therapeutic compound to be administered and the compound's concentration in the topical formulations depend upon the vehicle selected, the clinical condition of the patient, the side-effects and the stability of the compound in the formulation. Thus, the physician employs the appropriate preparation containing the appropriate concentration of the therapeutic compound and selects the amount of formulation administered, depending upon clinical experience with the patient in question or with similar patients.

The concentration of the therapeutic compound for topical formulation is in the range of about 0.01 mg/ml to about 100 mg/ml. Typically, the concentration of the therapeutic compound for topical formulation is in the range of about 0.5 mg/ml to about 50 mg/ml. The transepidermal flux rate of buspirone (individual or mean data) should be in one or more of the following ranges:
0.1 to 1.1 μg/cm²/hour as assessed over 5 hours following application;
0.09 to 0.60 μg/cm²/hour as assessed over 13 hours following application;
0.09 to 0.48 μg/cm²/hour as assessed over 24 hours following application;
0.08 to 0.46 μg/cm²/hour as assessed over 30 hours following application; and
0.08 to 0.39 μg/cm²/hour as assessed over 48 hours following application.

Solid dispersions of the therapeutic compound as well as solubilised preparations can be used. Thus, the precise concentration is subject to modest experimental manipulation in order to optimize the therapeutic response. Suitable vehicles include oil-in-water or water-in-oil emulsions using mineral oils, petrolatum and the like, as well as gels such as hydrogels suitable formulations may be oil or water-based, and include creams, lotions, ointments etc.

The therapeutic compound is optionally administered topically by the use of a transdermal therapeutic system (see Barry, Dermatological Formulations, Marcel Dekker, 1983, p. 181 and literature cited therein). While such topical delivery systems have been designed largely for transdermal administration of low molecular weight drugs, by definition they are capable of percutaneous delivery. They may be readily adapted to administration of the therapeutic compounds of the invention by appropriate selection of the rate-controlling microporous membrane.

The following Examples illustrate the invention.

### EXAMPLE 1

Formulation

A formulation of buspirone hydrochloride was prepared, additionally containing glycerol stearate, cetyl alcohol, PEG-100 stearate, white soft para isopropyl myristate, sorbitol, benzyl alcohol and purified water (composition: buspirone hydrochloride 5.5%, glycerol stearate 3%, cetyl alcohol 2.3%, PEG-100 stearate 2.3%, white soft paraffin 7.6%, isopropyl myristate, 4.5%, sorbitol 3.8%, benzyl alcohol 1% and purified water 70%). The flux rate of buspirone through human epidermis of this formulation was defined in vitro and the formulation subsequently tested in a clinical trial for efficacy in patients with atopic dermatitis. The formulation with its defined flux rate was found to be efficacious in reducing the extent of the atopic dermatitis and pruritis but did not result (when applied to 15% or less of total body surface area) in evidence of buspirone overdosage (combined symptoms of sedation, dizziness, gastric discomfort, nausea).

Buspirone Flux Rate Through Human Epidermis In Vitro

The in vitro human epidermal skin penetration of a topical preparation containing buspirone was studied using the Franz cell method (Howes et al., 1996, Methods for assessing percutaneous absorption. ECVAM Workshop Report ATLA 24 81). Skin from female cosmetic abdominoplasties (Europid, aged 30-45 years) was defatted and the full thickness skin stored at −20° C. until use. Epidermal sheets were separated from the dermis by immersing the skin in water at 60° C. for 45 seconds (Kligman et al, Arch. Dermatol. 88, 702-709, 1963). After this procedure, the skin was pinned down and the epidermal layer removed by gently separating the dermis and epidermis with blunt rat-toothed forceps.

The epidermis was floated onto a trough of distilled water and taken up onto filter paper supports and blotted dry with tissue paper. The skin was then positioned between the two halves of a Franz diffusion cell (exposed skin surface area=ca. 0.5 cm², receptor phase volume=approximately 1.7 ml but determined individually for each Franz cell), with the stratum corneum facing uppermost. These two halves were then clamped together.

Buspirone concentrations were determined using an HPLC method described below, although any appropriate method could be used. The HPLC instrument consisted of a Waters 717 plus Autosampler, Waters 2487 Dual λ Absorbance Detector, Waters 600 Controller Pump and Millenium Chromatographic Manager Software. The chromatographic conditions were column Hichrom 5μ C18 ODS column, length 250×4.6 mm, temperature 40_C, mobile phase 40% KH2PO4 (1.36 g/L) adjusted to pH 6.9 with 10% NaOH, 60% methanol:acetonitrile, 17:13, flow rate 1.0 ml/min, uv wavelength 210 nm, injection volume 20 μL, run time 30 nm.

Accurate determination of the rate of transport of buspirone through the skin relies upon the maintenance of sink conditions in the receiver fluid. The solubility of buspirone hydrochloride in phosphate buffered saline was found to be greater than 3 mg/ml. Such high solubility was sufficient to ensure sink conditions for the study. It was also established that buspirone was stable in the receiver fluid for the length of the study.

Buspirone cream formulation was applied to the surface of the skin at a target dose of 5 mg/cm² (Howes et al, 1996), and rubbed in using an applicator with ten circular motions in both clockwise and anti-clockwise directions. To determine the dose delivered, approximately 4 mg of cream was accurately weighed onto the tip of the applicator. The dose was then applied as described above onto a piece of control epidermal sheet and the applicator was weighed after application to account for any loss of formulation during the procedure. This was repeated ten times and the mean (3.0±0.1 mg of formulation/cell) was taken as the dose deposited for the Franz cell studies, which equated to an average buspirone hydrochloride dose of 197.1 μg (equates to 175.1 μg as free base). The receptor chamber of each cell was filled with PBS (phosphate buffered saline). Twelve replicates from three donors for each formulation (3×4 samples) were prepared. The Franz cells were immersed in a constant temperature water bath such that the receptor chambers were maintained at 37.0±0.5° C. throughout the experiment. This ensured that the skin surface temperature was maintained at 32.0±1° C. The receptor chamber contents were continuously agitated by small PTFE-coated magnetic followers driven by submersible magnetic stirrers.

A sample (200 μL) of the receptor phase was removed at 1, 5, 13, 24 and 30 hours (immediately replaced with fresh receiver fluid, PBS) with the final sample taken and the mass balance performed at 48 hours as per ECVAM guidelines (Howes et al, 1996). These samples were analysed for buspirone content by high performance liquid chromatography (HPLC) as described above and the amount and percentage permeated were plotted against time.

The minimum and maximum flux rates for buspirone across the epidermal layers in the Franz cell were determined at 5, 13, 24, 30 and 48 hours after starting the experiment (set of nine experiments). The results are tabulated below.

| | Maximum | Minimum |
|---|---|---|
| | flux | flux |
| Hour | (μg/cm²/h) | (μg/cm²/h) |
| 5 | 1.07 | 0.1 |
| 13 | 0.6 | 0.09 |
| 24 | 0.48 | 0.09 |
| 30 | 0.46 | 0.08 |
| 48 | 0.39 | 0.08 |

### EXAMPLE 2

Efficacy Study in Patients with Mild to Moderate Atopic Dermatitis and Pruitis

A randomised, double-blind, placebo-controlled, parallel group study was conducted in 82 patients with mild to moderate atopic dermatitis (AD) including some patients with pruritis. The patients received either the test medication (topical cream containing buspirone hydrochloride 5.5%, as described in Example 1) or reference medication (in this case placebo, topical cream without buspirone, composition glycerol stearate 3.8%, cetyl alcohol 2.9%, PEG-100 stearate 2.9%, white soft paraffin 9.3%, isopropyl myristate 5.6%, sorbitol 4.7%, benzyl alcohol 1% and purified water 70%) as determined by randomisation. During the 4-week treatment phase, the test medication or placebo was applied twice daily to all skin areas affected by atopic dermatitis that required treatment. The treatment phase was preceded by a wash-out period of at least 3 days without treatment.

The severity of the AD was assessed at screening, at the end of the wash-out phase, i.e. before the first application of study medication, and on Days 15 and 29 of the treatment phase by the investigator using the standardised scoring index SCORAD (Scoring in Atopic Dermatitis). Itching was rated twice daily by the patients themselves using a visual analogue scale (VAS). For this, the patients were asked to grade the current itching at the target area on a 10 cm visual analogue scale. The ends of the scale were labelled no itching (corresponding to 0 cm) and worst possible itching (corresponding to 10 cm). Measurements were made immediately prior to each application of cream. Measurement at this time would reflect chronic itch and would not assess any immediate effects of the cream on itch. In addition to chronic itch, a possible effect on acute itching was assessed by taking measurements immediately prior to the first application of cream (on day 1) and at 1 hour, 2 hours, 3 hours, 6 hours as well as immediately before the second application on day 1 (nominally 12 hours after 1^{st} application).

The primary efficacy variable for atopic dermatitis was the SCORAD total score (cumulative index). The primary endpoint was the examination on Day 29. Secondary endpoints were the examinations at other visits. The difference to Baseline (Day 1) was used in the statistical analysis.

The results of the study showed that the total SCORAD score in an analysis of 68 patients (per protocol analysis) who used the test medication twice daily for 4 weeks for the treatment of atopic dermatitis was lower than the total SCORAD score for patients using placebo. Observing the change in mean total SCORAD score for baseline and day 29 values there was a 31% decrease with placebo (n=34) and a 49% decrease with test (n=30). There was a statistically significant difference for the total SCORAD score (% change to baseline) between test and placebo on day 15 in the per protocol dataset (p=0.0386).

The test medication significantly reduced chronic itch over the 29 day period which would be expected from a reduction in the severity of the atopic dermatitis. Surprisingly, however, it was found that there was a dramatic decrease in itch following on very rapidly (within 1 hour) from the first application of test cream. This acute reduction in itch was unexpected and indicates a direct action of the test cream on itch unrelated to any immuno modulatory properties of the drug. From the intention to treat data set the itch values (median score) for test were as follows:—before itch 3.5 cm, 1 hour after application 1.75 cm, 2 hours after application 1.15 cm, 3 hours after application 0.85 cm, 6 hours after application 1.05 cm and 12 hours after application 1.25 cm (n=37−38). Thus the anti itch effect was already manifest 1 hour after application with a 50% reduction in itch, the maximum effect occurred 3 hours after application after which there was some recovery of the effect. The 3 hour effect corresponded to a 76% decrease in median itch. For placebo the corresponding 3 hour value was only a 37% decrease (less than half the decrease seen with test, n=40).

None of the patients exhibited the range of effects attributed to buspirone overdosage (as defined above) during their period of treatment.

1. A liquid or semi-solid topical formulation of buspirone which, when applied to human epidermis in vitro, results in a transepidermal flux rate of buspirone in one or more of the following ranges:
0.1 to 1.1 μg/cm²/hour as assessed over 5 hours following application;
0.09 to 0.60 μg/cm²/hour as assessed over 13 hours following application;
0.09 to 0.48 μg/cm²/hour as assessed over 24 hours following application;
0.08 to 0.46 μg/cm²/hour as assessed over 30 hours following application; and
0.08 to 0.39 μg/cm²/hour as assessed over 48 hours following application.

2. The formulation according to claim 1, which is non-occlusive.

3. The formulation according to claim 1, which is a cream, ointment of gel.

4-11. (canceled)

12. A method for the treatment of pruritus or an immune-related skin disease in a human patient, which comprises topical administration to the patient of an effective amount of buspirone.

13. The method according to claim 12, for the acute treatment of pruritis.

14. The method according to claim 13, wherein the treatment is given for up to 72 hours.

15. The method according to claim 13, wherein the treatment is given for up to 24 hours.

16. The method according to claim 12, wherein the pruritis is not associated with an immune disorder.

17. The method according to claim 12, for the treatment of atopic dermatitis.

18. The method according to claim 12, for the treatment of psoriasis.

19. The method according to claim 12, wherein the buspirone is applied over the whole area of the condition being treated.

20. The method according to claim 12, wherein the buspirone is in a liquid or semisolid topical formulation which, when applied to human epidermis in vitro, results in a transepidermal flux rate of buspirone in one or more of the following ranges:
0.1 to 1.1 μg/cm²/hour as assessed over 5 hours following application;
0.09 to 0.60 μg/cm²/hour as assessed over 13 hours following application;
0.09 to 0.48 μg/cm²/hour as assessed over 24 hours following application;
0.08 to 0.46 μg/cm²/hour as assessed over 30 hours following application; and
0.08 to 0.39 μg/cm²/hour as assessed over 48 hours following application.

21. A cream, ointment or get comprising 0.5 to 50 mg/ml of buspirone.
